(19) United States
(12) Patent Application Publication
(10) Pub. No.: US 2015/0233751 A1
(43) Pub. Date: Aug. 20, 2015

(54) OPTICAL FILL DETECTION

(71) Applicant: KONINKLIJKE PHILIPS N.V., Eindhoven (NL)

(72) Inventors: Robert Lloyd Blake, Cambridge (GB); Nicholas Young Kent, Sandhurst (GB)

(73) Assignee: KONINKLIJKE PHILIPS N.V., EINDHOVEN (NL)

(21) Appl. No.: 14/434,792

(22) PCT Filed: Oct. 12, 2013
(86) PCT No.: PCT/IB2013/059330

§ 371 Date: Apr. 10, 2015

Related U.S. Application Data
(60) Provisional application No. 61/713,030, filed on Oct. 12, 2012.

(51) Int. Cl. G01F 23/292; A61B 10/00

(52) U.S. Cl. CPC G01F 23/292; A61B 10/007

(57) ABSTRACT

The present invention relates to the field of home monitoring. In particular the present invention relates to a device for the analysis of sample material, comprising a sample container, wherein said sample container is configured for holding sample material, a light source, wherein said light source is configured for irradiating the sample container a detector, wherein said detector is configured to detect light from the sample container in response to an irradiation of the sample container by the light source and an assessment unit for assessing the fill level of the sample container based on the detected light, as well as the use of such a device for home monitoring parameters of a bodily fluid of a subject. The present invention further relates to a method for assessing the fill level of a sample container configured for holding sample material.

### FIELD OF THE INVENTION

The present invention relates to the field of home monitoring. In particular the present invention relates to a device for the analysis of sample material comprised in a sample volume, comprising a light source, wherein said light source is configured for irradiating the sample volume; a detector, wherein said detector is configured to detect light from said sample volume in response to an irradiation of said sample volume by the light source; and an assessment unit for assessing the fill level of said sample volume based on the detected light. The present invention further relates to a sample container configured for holding sample material in a sample volume comprising a valve configured for moving said sample material, wherein said valve is configured for changing the direction of at least part of impinging light upon irradiation of the sample volume. The present invention also relates to a method for assessing the fill level of a sample volume configured for holding sample material.

### BACKGROUND OF THE INVENTION

The analysis of blood, e.g. the determination of the amount of white blood cells, or red blood cells, is an activity which is typically carried out in a hospital, or a laboratory by a medical professional. However, due to improvements in the development of mobile analysis devices and the advent of suitable telemedicine solutions, home monitoring of parameters of bodily fluids has become feasible. Accordingly, patients themselves—without the direct assistance of medical professionals—can use integrated devices in order to check parameters of blood, urine or other bodily fluids on a monthly, weekly, daily or even an hourly basis. Such analyses are particularly helpful in case of chemotherapy regimens, in which bone marrow activity is inhibited, leading to a decreased production of blood cells and platelets. Patients with low blood cell counts are in danger of serious complications from an infection, as well as not being able to receive their next treatment due to low cell counts. Corresponding results on blood cell levels or other bodily fluid parameters can then be transmitted to healthcare professionals allowing for telemedical or direct intervention.

In order to become suitable for such an approach home monitoring devices have to be robust and as fail safe as possible. It should, in particular be avoided to produce false results, or to force the patient to repeat measurement steps due to a maloperation of the device. A typical problem, which frequently occurs during the handling of such devices, is their failing due to incorrect filling, e.g. with a bodily fluid such as blood or urine. An accurate analysis of bodily fluid parameters requires a correct filling of the analysis device since wrong volumes or the presence of air bubbles would lead to unusable or incorrect results. Furthermore, incorrect fillings of a device which are not detected before starting the analysis of the bodily fluid may lead to an increased failure rate of the testings and an increased number of test repetitions thus raising the associated operation costs.

In consequence there is a need for the development of a sample material monitoring device which is capable of reducing the test failure rate and which is suitable for home use by the patient.

### OBJECTS AND SUMMARY OF THE INVENTION

The present invention addresses these needs and provides devices comprising an automatic fill detection feature. The above objective is in particular accomplished by a device for the analysis of sample material a device for the analysis of sample material comprised in a sample volume, comprising a light source, wherein said light source is configured for irradiating the sample volume; a detector, wherein said detector is configured to detect light from said sample volume in response to an irradiation of said sample volume by the light source; and an assessment unit for assessing the fill level of said sample volume based on the detected light. It was in particular found by the inventors that a device as described allows to prevent a test being performed when it is already known that the test will fail with high likelihood. A detection of filling problems, e.g. due to the presence of voids or air bubbles, can advantageously be displayed to the user to inform her or him that sample volume has not been filled correctly and that intervention is required, e.g. by a repetition of the filling activity.

In a preferred embodiment of the invention the sample volume is comprised in said device.

In a further preferred embodiment of the present invention the sample volume is comprised in a separable sample container.

In a preferred embodiment of the invention the detector of the device is configured for detecting light transmitted through the sample volume.

In a further preferred embodiment said light source is configured to emit light having a wavelength of 475-575 nm or 260-350 nm. The use of green light in the range of 475-575 nm provides the advantageous effect that substances which absorb light of this wavelength range can be detected. Red blood cells, in particular hemoglobin absorbs light of the indicated range quite well. Similarly, further bodily fluids such as urine can be detected, e.g. by the use of different wavelength in the range of 260-350 nm. Urine accordingly absorbs light having this wavelength. The presence of blood via the absorption of light by hemoglobin or the presence of urine via the absorption of urea or uric acid can accordingly be detected. The detection of such absorbances accordingly allows for the determination whether a correct filling is given in the sample volume or not.

In a further embodiment the device additionally comprises an optical element configured to image at least a part of the sample volume onto the detector.

In yet another embodiment said optical element as mentioned herein is or comprises a lens, is or comprises a mirror, and is or comprises an optical fiber. Further envisaged are combinations of lenses, one or more mirrors, and/or one or more optical fibers.

In yet another preferred embodiment of the present invention the device as mentioned herein above comprises scanning means configured to detect light from the entire sample volume. The detection of light from the entire sample volume provides the advantageous effect that potential blind spots at positions of air bubbles or other filling discrepancies are largely avoided. It is preferred that the scanning means to be used is a camera.

In another preferred embodiment, said assessment of the fill level of the sample volume comprises the assessment of the presence of a void. A typical example of a void is an air bubble, which should be detectable upon the employment of the above described structures of the device.

In a further aspect the present invention relates to a sample container configured for holding sample material in a sample volume comprising a valve configured for moving said sample material. Within the sample container sample material such as blood or other bodily fluids may accordingly be transported from an opening section to a testing or control section, or from a top position to a bottom position. The valve may have suitable forms in order to allow for an optical detection of the fluid transport process in the sample volume. It is preferred that said valve is configured for changing the direction of at least part of impinging light upon irradiation of the sample volume. In a further preferred embodiment of the invention the valve comprises a rounded edging or a radial edging. Blind spots may be present at sites of non-optimal fillings, e.g. due to the presence of voids or gaps in the bodily fluid to be tested and lead to a potential failure to detect non-correct filling states of the sample volume within the sample container. Since blind spots typically occur in the valve section of the sample container, a rounded or radial edging of the valve advantageously reduces the maximum area of blind spots due to increased optical detection.

In another preferred embodiment a valve as mentioned herein above comprises polished surface material.

In yet another preferred embodiment of the invention the valve as mentioned herein above is transparent. A transparent valve may comprise or is made of transparent plastic material. In a particularly preferred embodiment said valve comprises or is made of polycarbonate material. The transparency of valve material was found to further increase the optical properties of the valve and thus the image quality. It was accordingly found that the use of transparent material, such as polycarbonate material, advantageously improves image quality and reduces the maximum area of blind spots due to increased optical detection.

In a further aspect the present invention relates to a system comprising a device as defined herein above and a sample container as defined herein above. It is preferred that the sample container is or is comprised in a cartridge and that the light source, detector and assessment unit are comprised in a reader or control unit. Accordingly, the cartridge is configured to be processed by said reader. The cartridge advantageously has a form or configuration which fits into the reader, which may provide an opening or receptacle structure for the cartridge.

In a preferred embodiment said sample material may be any bodily fluid. It is particularly preferred that the sample material is blood or urine.

In another aspect, the invention relates to a method for assessing the fill level of a sample volume, wherein said sample volume is configured for holding sample material, comprising:

irradiating the sample volume with light having a wavelength of 475-575 nm or 260-350 nm;

detecting light transmitted through the sample volume in response to said irradiation; and

assessing the fill level of the sample volume based on the detected light.

This method advantageously allows detecting the presence of bodily fluids or substances which absorb light of the wavelength range of 475-575 nm or 260-350 nm. Among bodily fluids which are detectable by irradiation with a wavelength of 475-575 nm is blood comprising hemoglobin which absorbs light of the indicated range quite well. Similarly, further bodily fluids such as urine can be detected, e.g. by the use of different wavelengths in the range of 260-350 nm. The detection of such absorbance in the indicated wavelength ranges accordingly allows for the assessment of the fill level of the sample volume.

In a preferred embodiment said wherein said assessment of the fill level is carried out with an image processing algorithm comprising the steps:

acquiring an image of the sample material;

checking the focus of the image;

optionally adjusting the focus of the image;

COM calculating a region of interest;

vertical line scanning to determine the sample material centre and sample material rotation;

horizontal line scanning to determine the presence of a void;

optionally calculating the size and/or volume of the void; and

optionally recalculating the fill level of the sample volume.

In another embodiment the present invention relates to the use of a device or system as mentioned herein above for home-monitoring parameters of a bodily fluid of a subject. In a preferred embodiment, a device or system as mentioned herein above is used for home-monitoring parameters of blood or urine.

### BRIEF DESCRIPTION OF THE DRAWINGS

FIG. 1 schematically shows a cartridge for a device comprising an automatic fill detection feature according to the present invention.

FIG. 2 shows the filling state of a cartridge for a device comprising an automatic fill detection feature according to the present invention. The cartiridge is considered to be correctly filled when the blood is in the valve (B) and in control area (C).

FIG. 3 schematically shows a system for automatic detection of levels in a cartridge according to the present invention.

FIG. 4 shows a flow diagram of an image processing algorithm according to an embodiment of the invention.

FIG. 5 schematically shows a cross-section through a valve according to an embodiment of the invention.

FIG. 6 schematically depicts light being bent by a valve structure comprising a lens according to an embodiment of the present invention.

FIG. 7 depicts an air bubble in the visible part of a valve.

### DETAILED DESCRIPTION OF THE EMBODIMENTS

The present invention relates to a device for the analysis of sample material allowing to automatically detect filling states of a sample volume, a sample container configured for holding sample material, as well as corresponding methods and uses.

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect a device for the analysis of sample material comprised in a sample volume, comprising a light source, wherein said light source is configured for irradiating the sample volume; a detector, wherein said detector is configured to detect light from said sample volume in response to an irradiation of said sample volume by the light source; and an assessment unit for assessing the fill level of said sample volume based on the detected light.

The term “device” as used herein refers to a structure or an instrument, or part of an instrument, or a part of a system, which allows or is suitable for the performance of reactions, in particular molecular reactions involving chemical and/or biological entities and/or particle counting activities and/or the measurement of physical and/or chemical parameters. The device may correspondingly be equipped, for example, with one or more inlet and/or outlet elements, it may comprise one or more surfaces, e.g. reactive surfaces or surfaces with specific functionality, it may comprise a reaction zone, a washing zone, a mixing zones, a waiting zone, a measurement zone, a waste zone, a reservoir zone, a recollection or a regeneration zone etc. or any sub-portion or combination thereof. It may further comprise units allowing the counting of cells or particles in a bodily fluid. For example, the device may be configured to count particles according to suitable principles e.g. according to the Coulter principle. The counted particles may, in specific embodiments, be white blood cells, red blood cells, inorganic particles, bacterial cells etc. The device may further comprise connections between the mentioned elements, e.g. tubes or joints; and/or it may comprise reservoirs and repositories for liquids, fluids, chemicals, ingredients, samples or any other entity to be used within the device. In further embodiments of the invention the device may additionally or alternatively comprise heating modules or regulating units for controlling and/or regulating the temperature, e.g. a heating zone wherein the temperature may be kept constant at a desired value, or may be set to a desired value in dependence of a reaction type or reaction cycle etc. In further embodiments the device may additionally or alternatively comprise cooling modules, e.g. a cooling zone wherein the temperature may be kept constant at a desired value, or may be set to a desired value in dependence of a reaction type or reaction cycle etc. These zones may further also be equipped with suitable sensor elements allowing the measurement of temperature changes or temperature gradients.

The device may additionally comprise units or elements configured to measure the temperature of a sample, the increase or decrease of temperature over a period of time, the pH of a sample, ionic concentrations in a sample, or may detect the presence of molecules in a sample, e.g. the presence of proteins, organic small molecules, nucleic acids etc.

Additionally or alternatively, the device may comprise units, elements or equipment allowing to change further parameters such as the presence of charged entities, the presence of ions, or may convey mechanical or shearing forces etc. For example, the element(s) may be suited to establish an electric or electrophoretic current, the element(s) may be suited to provide a specific pH or a specific presence of chemical or physical entities, e.g. the presence of certain acids, salts, ions, solvents etc. and/or the element(s) may be suited to provide a strong medium movement. Any of the above mentioned additional facilities may be available in any part of a device, e.g. in a reaction zone or reaction chamber.

In further specific embodiments the device may additionally or alternatively comprise modules allowing the detection of flow velocity, viscosity or density values, the transition of one state to another, the presence or absence of reagents etc.

Furthermore, the device may comprise an electronic or computer interface allowing the control and manipulation of activities in the device, and/or the detection or determination of reaction outcomes or products. The device may accordingly be equipped with wireless connectivity to a second device, e.g. a user interface unit, allowing the transfer of acquired data from the device to the user interface. Said user interface, which may additionally be equipped with different communication and/or calculating functionalities may function as telehub for a remote patient record system, e.g. in a hospital or in a medical practice.

Furthermore, the device may, in certain embodiments, contain openings or entrance holes in order to allow insertion of elements or device parts. It is further envisaged that such entrance holes or openings may be provided with lids, caps or closure heads. In further embodiments, the device may be provided in a light tight form. I.e. the device may be configured to eliminate the entrance of ambient light. For example, openings or entrance holes may be shut light tight. Alternatively, a housing or casing for a device may be provided, thus allowing the performance of filling control and further analytical steps in the absence of ambient light.

According to the invention the device is for the analysis of sample material which is comprised in a sample volume. The term “sample volume” refers to a space, cavity or room which can be filled with sample material, e.g. a liquid sample. Such sample material may be hold in said volume and/or can be moved through said volume, e.g. to certain zones or regions of the device, or zones or regions outside of the device. The sample volume may have any form or size. The volume may be or comprise a capillary tube, allowing for capillary motion of sample material. The volume may, in alternative embodiments, be of a larger size and accordingly require gravity, or mechanical or electrochemical forces for sample material transport. The sample volume may be adapted to the sample material to be analyzed, e.g. to the typical amount of sample necessary for diagnostic or medical analysis. In further embodiments, the adaptation may be achieved via an active adaptation process at the device, e.g. an increase or decrease of the space available. It is preferred that the sample volume is optically accessible in order to allow for a determination of its filling state. Such optical accessibility may be achieved by transparent or semi-transparent wall structures.

In a specific embodiment of the present invention said sample volume may be comprised within said device. The device may accordingly comprise, besides a light source, a detector and an assessment unit, a space, cavity, room or receptacle which can be filled with sample material. The sample volume may be physically linked to device entities, e.g. a light source, a detector or an assessment unit and be considered as integral part of the device. In certain embodiments, a sample volume may be simply structured, i.e. it may be based on capillary tubes allowing for capillary movements of sample material. Alternatively, it may comprise additional elements for the transport and distribution of sample material, e.g. pumps, valves, electrochemical elements etc.

A sample volume being comprised in a device according to the present invention may, in certain embodiments, comprise an opening allowing for direct sample taking, e.g. blood, or may be configured to allow the introduction of additional medical equipment, e.g. syringes, pipettes or interface units of automated sample taking devices. For example, for the analysis of blood a sample volume or internal receptacle may have a specifically adapted opening allowing the taking of blood samples. For the analysis of urine samples, the opening may be connected to a funnel element, or an exterior tubing.

In order to be able to hold the sample material, the sample volume may be configured to be impermeable with regard to additional zones or units of the device, or further elements of the device. Such an impermeability may be transitory or may be locally removable, e.g. by opening gates or openings within the separable sample container and/or within the device.

In another specific embodiment of the present invention said sample volume may be comprised in a separable sample container. The term “separable sample container” refers to an entity which is not physically linked to a device comprising a light source, a detector and an assessment unit. The separable sample container may, in certain preferred cases, fit into the device and/or be recognized, actuated, assessed or controlled by the device.

The separable sample container is configured to hold within the sample volume sample material. In further embodiments, the sample container is configured to move said sample material through the sample container, e.g. to certain zones or regions of the sample container, or to zones or regions outside of the sample container, e.g. to zones or regions of the device. A separable sample container according to the present invention may comprise an opening allowing for direct sample taking, e.g. blood, or may be configured to allow the introduction of additional medical equipment, e.g. syringes, pipettes or interface units of automated sample taking devices. For example, for the analysis of blood a sample container may have a specifically adapted opening allowing the taking of blood samples. For the analysis of urine samples, the opening may be connected to a funnel element, or an exterior tubing.

In order to be able to hold the sample material, the sample container may be configured to be impermeable with regard to zones, regions or units of the device, or further elements of the sample container. In specific embodiments the sample volume of the sample container may be configured to be impermeable with regard to additional structural elements of the sample container and/or with regard to additional structural elements of the device. Such an impermeability may be transitory or may be locally removable, e.g. by opening gates or openings within the separable sample container and/or within the device. A separable sample container according to the present invention may further comprise elements allowing the movement of the sample, e.g. towards reaction or assessment zones. Furthermore, the separable sample container may be equipped with tubes or channels. For the analysis of bodily fluids different types or configurations of sample container may be provided. For example, for the analysis of blood a sample container may have specifically adapted opening allowing the taking of blood samples. For the analysis of urine samples, the opening may be connected to a funnel element, or an exterior tubing.

According to the invention the device as defined herein above further comprises a light source. The term “light source” as used herein refers to an illumination source including photo-luminescent sources, fluorescent sources, phosphorescence sources, lasers, electro-luminescent sources, such as electroluminescent lamps, and light-emitting diodes. The term “light-emitting diode” as used herein refers to any system that is capable of receiving an electrical signal and producing a color of light in response to the signal. The light emitting diodes accordingly include light-emitting diodes (LEDs) of all color types, e.g. white LEDs, ultraviolet LEDs, visible color LEDs, infra-red LEDs, light-emitting polymers, organic LEDs, electro-luminescent strips, silicon based structures emitting light etc. Among the visible color LEDs green light LEDs, red light LEDs, blue light LEDs, yellow light LEDs etc. may be used. The light source is specifically configured for irradiating the sample volume.

The light source may further be a combination of different LEDs, e.g. of different visible color LEDs. The light source may also be equipped with energy or intensity regulators allowing for the adjustment of the amount of light energy being emitted.

In specific embodiments, the light source may additionally be combined with filter elements allowing for reduction or modification of the emitted wavelengths, or wavelengths spectrum.

The light source may further be combined with additional optical, opto-mechanical or mechanical elements. For example, a diffuser may be placed in the light path between the light source and the sample volume or sample. A diffuser is a device that diffuses or spreads out or scatters light in some manner, in particular to give soft light. Diffuse light can be obtained, for example, by making light to reflect diffusely from a white surface. Furthermore, compact optical diffusers may use translucent objects, and can include ground glass diffusers, teflon diffusers, holographic diffusers, opal glass diffusers, and/or greyed glass diffusers. Furthermore, lens systems, focusing systems, optical limitations avoiding unintended radiation of light etc. may be placed in the light path between the light source and the sample volume or sample material. Furthermore, a wall element may be placed in the light path, in particular a wall element delimiting the sample volume from a reader or control unit. The wall element is preferably transparent allowing for efficient separation between the sample volume and any electrical or mechanical element of the device and at the same time allowing for the transmission of light from the light source to the sample.

Accordingly, the light source or light sources may be positioned in way that light is transmitted from the source(s) directly or indirectly towards the sample volume, comprising, for example, a blood or urine sample. The light path may be direct, i.e. leading from the light source without interruptions and bendings to the sample volume. The light path may alternatively be interrupted by additional elements such as filters or diffusers, or may be bent, e.g. by the use of optical fibers or mirror systems.

According to the invention the device further comprises a detector. The term “detector” as used herein refers to an optical sensor, which converts an optical signal into an electrical signal. Such detector may, for instance, be located in a reader and control unit, whereas the sample material may be located in an analyzing or reaction unit functioning as sample container. Alternatively, the detector may be provided in the same device where the sample material is present. The detector is positioned in the light path after the sample volume, i.e. detecting light which has passed the sample volume and thus potentially also the sample material. In a preferred embodiment, the detector is configured to detect light which is transmitted through the sample volume. Accordingly, the detector is preferably used to detect transmitted radiation, i.e. is placed at the opposite of the starting point of the light path starting at the light source and going through the sample volume and potentially also the sample material, i.e. if sample material is present. The term “transmission” as used herein means the property of a substance, e.g. a sample material, to permit the passage of light, with some or none of the incident light being absorbed in the process. It is understood that if some light is absorbed by the substance, e.g. sample material, then the transmitted light will be a combination of the wavelengths of the light that was transmitted and not absorbed. A detector according to the current invention may accordingly detect the absorbance of light by a substance in the light path, e.g. by the sample material such as blood or urine. The detection may, in specific embodiments, comprise the detection of specific wavelengths, or wavelengths ranges. Furthermore, the detected wavelengths or wavelengths ranges may be compared to the wavelengths or wavelengths ranges emitted by the light source to determine which wavelengths can be transmitted and which are absorbed by the sample, e.g. blood or urine. Measurement values obtained may be transferred to an assessment unit. In alternative embodiments, values obtained may be accumulated and/or saved in a storage medium, e.g. a USB stick or hard drive, or they may be transferred to a user interface or medical or hospital system, e.g. over wireless interaction.

According to the invention the device further comprises an assessment unit for assessing the fill level of the sample volume based on the detected light. The “assessment unit” may be an electronic circuit integrating data from the light source, e.g. concerning the emitted light wavelengths, and data from the detector concerning the absorbed wavelengths, and/or the wavelengths and light intensity transmitted through the sample. The assessment unit may accordingly calculate the degree of transmission of light. The assessment unit may further compare the obtained information with comparison values or control values. The assessment unit may accordingly be provided or comprise threshold values indicating whether the measured value reflects a correct filling, or an incorrect filling, e.g. due to the presence of voids or air bubbles, on the basis of comparisons between the detected transmission of light or absorbance of light and corresponding control values.

In a particularly preferred embodiment, the assessment of the fill level of the sample volume therefore comprises the assessment of the presence of a void. A typical cause for a void is the inclusion of an air bubble in the sample. The assessment thus aims at identifying air bubbles in the filled sample volume, i.e. in the sample and to inform the user about their presence, thus allowing for a rapid termination of the analysis program without performing subsequent molecular analysis steps, although a failure of such steps is to be expected.

In specific embodiments of the present invention, the threshold value for incorrect filling may be a deviation of about more than 20%, more than 15%, more than 14%, more than 13%, more than 12%, more than 11%, more than 10%, more than 9%, more than 8%, more than 7%, more than 6%, or more than 5% from a complete filling state, such as a complete filling state with a normal sample of the same type, e.g. via a control value obtained with a correctly filled sample volume comprising a normal sample of the same type. The term “normal sample of the same type” refers to a sample having the same origin and approximately the same consistency as the sample tested. In further embodiments, the control sample may be obtained from a healthy individual showing no phenotypic alterations in the sample type tested. For example, if blood samples are tested, the normal sample of the same type is a blood sample derived from a healthy individual not afflicted by blood diseases, anemia or other blood consistency modifying conditions. If, for example, urine samples are tested, the normal sample of the same type is a urine sample derived from a healthy individual not afflicted by kidney or urogenital diseases or other urine consistency modifying conditions.

In very specific embodiments, in case of previous knowledge of specific conditions, e.g. a previously known anemia of a patient, a different threshold value may be used. Accordingly, in specific embodiments of the present invention, the threshold value for incorrect filling may be a deviation of about, more than 20%, more than 15%, more than 14%, more than 13%, more than 12%, more than 11%, more than 10%, more than 9%, more than 8%, more than 7%, more than 6%, or more than 5% from a complete filling state with a disease sample of the same type, e.g. via a control value obtained with a correctly filled sample volume comprising a disease sample of the same type. The term “disease sample of the same type” refers to a sample having the same origin and approximately the same consistency as the sample tested. Furthermore, the control sample obtained from an individual showing phenotypic alterations in the sample type tested, e.g. anemia, or urine modifying diseases. For example one or more control values, which were previously derived from anemic or otherwise sick patients and/or are stored in the assessment unit or associated storage medium, may be used for the calculation of threshold values.

The assessment unit may further be connected to a display on the device. The assessment unit may accordingly provide data to be presented on a display. For example, should the calculation of transmission values result in an incorrect filling, the display may inform the patient about the result and request him to abort further testing and/or restart the testing with a different sample. The assessment unit may further be connected to an audio unit allowing to alert the patient about incorrect fillings via sound effects.

The device, e.g. reader, or a user interface may additionally be provided with or store information on the patient or user, for example, derived form of a personalized medical archive or an electronic health card. The device according to the present invention, e.g. reader, or a user interface may, for example, comprise a card reader or card adapter module which is configured to read electronic health cards or other types of personalized medical archives. In an alternative or additional embodiment, corresponding information may be transferred to the device from the patient's hospital record or healthy record previous to the testing. Such a transfer may be carried out via connection over the internet. Wireless connection to a user interface may be used to accomplish such a data transfer. The transfer of information may further be used in order to confirm and/or update information provided in a patient's personalized medical archive or electronic health card. This information may used for assay result verification, comparison purposes, connectivity purposes (e.g. to a hospital) or for documentation purposes.

In a specific embodiment of the invention the light source is configured to emit light having specific wavelengths. In one embodiment the emitted light wavelength is 475-575 nm, e.g. light having a wavelength of 475 nm, 480 nm, 485 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm or any wavelength in between the indicated values may be emitted. The wavelength emitted may also comprise a sub-range of the mentioned range, e.g. 500-560 nm, 510-550 nm, 520-550 nm, 530-550 nm, 500-530 nm, 500-540 nm etc. The emitted wavelengths in the indicated range of 475-575 nm essentially comprises green light. Green light is typically absorbed by hemoglobin molecules. The accordingly emitted wavelengths may, in specific embodiments, be used for the detection of the presence of hemoglobin in the sample container, i.e. in case of the analysis of blood samples.

The present invention also envisages that the light source is configured to emit light having wavelengths of further ranges within the visible spectrum, as well as the ultraviolet and infrared spectrum. In one embodiment, the emitted light wavelength may be 260-350 nm, e.g. light having a wavelength of 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm or any wavelength in between the indicated values may be emitted. The wavelength emitted may also comprise a sub-range of the mentioned range, e.g. 270-330 nm, 280-340 nm, 290-350 nm, 300-350 nm, 310-350 nm, 320-350 nm, 260-300 nm, 260-310 nm etc. The emitted wavelengths in the indicated range of 260-350 nm essentially comprise ultraviolet and violet light. This light is typically absorbed by urea and uric acid molecules. The accordingly emitted wavelengths may, in specific embodiments, be used for the detection of the presence of urea and uric acid molecules in the sample volume, i.e. in case of the analysis of urine samples.

For further samples additional wavelengths or wavelengths ranges may be used. The wavelength or wavelength spectrum to be emitted may accordingly be determined according to optical properties of the sample to be analyzed, in particular absorption and light transmission properties. Such properties may be derived from suitable databases or literature sources known to the person skilled in the art.

The configuration to emit light in the mentioned wavelengths may be achieved by the use of light sources which emit light in the indicated wavelengths, e.g. LEDs as mentioned herein above, or by using filters or filter systems for one type of light source, allowing for the employment of a single light source for the testing of different sample types. Furthermore, both options may be combined, i.e. different color LEDs may be combined with filters or filter systems.

It is further envisaged in an embodiment of the invention that the device comprises an optical element which is configured to image at least a part of the sample volume onto the detector. The term “optical element” as used herein refers to an entity which is capable of imaging light, transmitting light form a source or start point to the detector, or modifying light, e.g. its wavelength, intensity, direction etc. form a source or start point to the detector. In preferred embodiments, the optical element is or comprises a lens or a lens system. A lens or lens system may accordingly be positioned in the light path originating from the light source and passing the sample. The lens or lens system may focus light into a detector or sensor. The lens or lens system may be configured to be adaptable to different light sources and/or wavelengths. The lens or lens system may further be combined with additional optical elements, e.g. filter elements. In another preferred embodiment, the optical element is or comprises a mirror. A mirror may be positioned in the light path originating from the light source and passing the sample in order to direct the transmitted light into a different direction, e.g. if the detector is not located in the light path originating from the light source. The device may also comprise a mirror system comprising more than one mirror, able to transmit the light into more than one direction. This could advantageously be used for the detection or transmission by more than one detector. Furthermore, mirrors or mirror systems may be combined with further optical elements, e.g. with filter elements or lenses or lens systems. For example, if a mirror is combined with a specific filter, only a defined wavelength or wavelength range may be transmitted to a detector. Such a detector may accordingly be adapted to the recognition of such a defined wavelength or wavelengths range, e.g. UV light or green light. Furthermore, mirrors or mirror systems may be combined with lenses or lens systems to allow a focusing of reflected light to the detector.

The device may alternatively or additionally comprises an aperture, i.e. an opening that determines the cone angle of a bundle of rays that come to a focus in the image plane of a detector as described herein. The aperture typically determines how collimated the admitted rays are and may have an impact on the sharpness of the focus at the image plane, e.g. at a detector as described herein. An aperture may be provided in a fixated form, or it may be moveable. Accordingly, an aperture may be provided as edge of a lens or mirror, or it may be provided as ring or other fixture holding an optical element in place. In specific embodiments, it may have the form and function of a diaphragm placed in the optical path to limit the light admitted by the system. It may, for example, be provided as iris diaphragm.

The device may, in addition or in the alternative, comprise an optical fiber, i.e. a preferably flexible, transparent fiber which may be made of glass, e.g. silica, or plastic. Optical fibers typically function as waveguide and thus transmit light between the two ends of the fiber. In specific embodiments, the optical fiber may include a transparent core surrounded by a transparent cladding material with a lower index of refraction. Light is typically kept in said core by total internal reflection. Examples of suitable optical fibers, which may be used in the context of the present invention, are multi-mode fibers (MMF), or single-mode fibers (SMF). Multi-mode fibers are preferred since they have a wider core diameter, and are normally used for short-distance links and for applications where high power must be transmitted. On optical fiber as mentioned herein may accordingly be positioned in the light path originating from the light source and passing the sample in order to direct the transmitted light into a different direction, e.g. if the detector is not located in the light path originating from the light source. The optical fiber may further be positioned in the light path in order to avoid radiant emittance in the device, which might influence its working or the measurement of parameters. An optical fiber as mentioned herein may further be combined with additional optical elements such as a lens or lens system, a mirror or mirror system or filters or filter systems as mentioned herein.

In a further embodiment of the present invention, a device according to the invention comprises scanning means configured to detect light form the entire sample volume. The term “scanning means” as used herein refers to any detector element as defined herein, which is capable of detecting the transmission of light through the entire sample volume. The detection of transmission of light through the entire sample volume allows to detect most or essentially all potential blind spots at positions of air bubbles or other filling discrepancies throughout the sample volume, thus avoiding filling artefacts in certain sample volume zones. The scanning means may, in one embodiment, be a detector as defined herein above.

The scanning means as envisaged by the present invention may preferably be provided in a bound or fixated position at the end of the light path. In an alternative embodiment, the scanning means may be moveable, e.g. along the light path after the transmission through a sample, or from a position above the sample volume into the sample volume or partially into the sample volume. Moving scanning means may provide the effect that the transmission of light can be determined at different locations of the sample volume, and/or that the field of vision is extended, thus excluding the presence or reducing the size of blind spots in the light path, e.g. caused by structural elements present in or forming the sample volume. In further embodiments the light source(s) may be moveable. Moving the light source(s) may provide the effect that the field of vision is extended, thus excluding the presence or reducing the size of blind spots in the light path, e.g. caused by structural elements present in or forming the sample volume.

It is preferred that the scanning means is a camera. Examples of suitable cameras in the context of the present invention are active-pixel sensors (APS), i.e. image sensors consisting of an integrated circuit containing an array of pixel sensors, each pixel containing a photodetector and an active amplifier. Examples of APS include CMOS sensors and charged couple device (CCD) image sensors. It is preferred that CMOS sensors are used for the detection of transmitted light.

In a particularly preferred embodiment of the invention, a small format CMOS sensor or camera may be used. It is further preferred that this camera is equipped with a wide angle lens.

In a further aspect the present invention relates to a sample container configured for holding sample material in a sample volume comprising a valve configured for moving said sample material. A sample container according to the present invention may accordingly hold, within a sample volume, sample material, e.g. as defined herein above in the context of a separable sample container. In order to be able to hold the sample material, the sample container may be configured to be impermeable with regard to further zones, regions or units of the sample container and/or with respect to outside elements.

The amount of sample material which can be filled into the sample container may vary, e.g. be in the range of 1, 10, 100 or 1000 μl, or in the range of 1, 10 or 20 ml. The amount may further be adapted to certain parameters, e.g. the sample material to be analyzed. For example, the typical amount of sample necessary for diagnostic or medical analysis may be used as benchmark value for the size of the sample volume in the sample container. In further embodiments, the adaptation may be achieved via an active adaptation process at the sample container, e.g. an increase or decrease of the space available may be accomplished by blocking cavities etc.

A sample container according to the present invention may further comprise elements allowing the movement of sample material, e.g. towards reaction or assessment zones. Furthermore, the sample container may be equipped with tubes or channels. For the analysis of bodily fluids different types or configurations of sample container may be provided. For example, for the analysis of blood a sample container may have specifically adapted opening allowing the taking of blood samples. For the analysis of urine samples, the opening may be connected to a funnel element, or an exterior tubing.

A sample container according to the present invention may further comprise an opening allowing for direct sample taking, e.g. blood, and/or may be configured to allow the introduction of additional medical equipment, e.g. syringes, pipettes or interface units of automated sample taking devices.

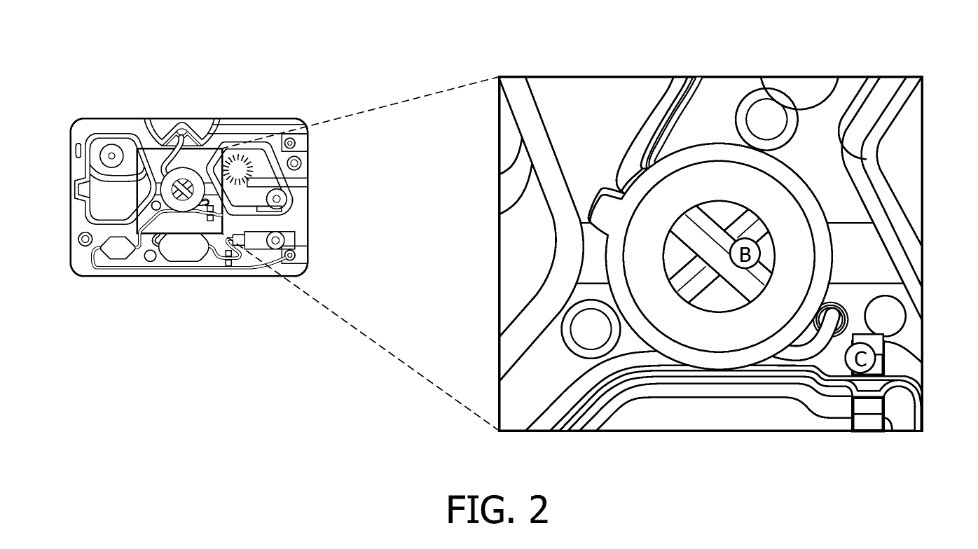

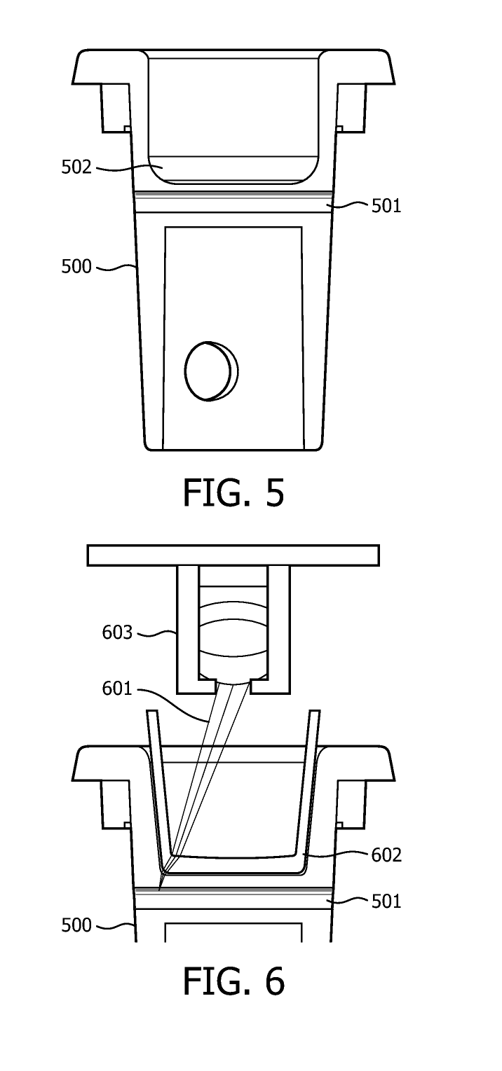

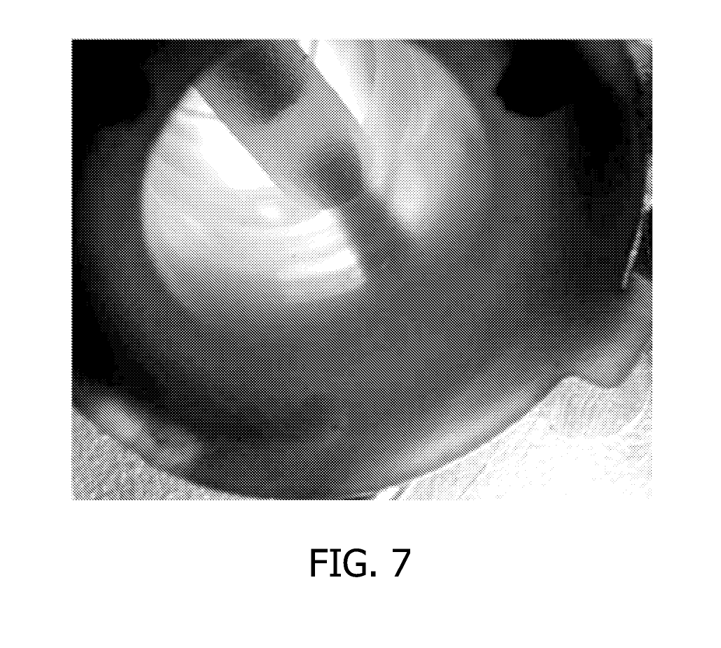

A “valve” as used herein refers to a central mechanical element which allows to move sample material, e.g. towards reaction zones of the sample container etc. and to control these movements. The valve preferably comprises a sample volume in the form of a capillary tube or channel, which can be filled with sample, e.g. blood or urine. The control of movements and the function of the valve may be initiated in a separable entity, e.g. an outside reader or a control unit. A valve may, for instance, be placed in the centre of the sample container. An example of a valve is shown in FIG. 2, FIG. 5 and FIG. 6. The form and configuration of the valve has been found by the inventors to contribute to the quality of the imaging process. According to embodiments of the invention, the valve is filled with sample during the analysis steps, e.g. in a capillary tube or channel, and may accordingly comprise filling discrepancies such as voids or air bubbles. In order to improve the detection of such filling discrepancies, it was found to be of importance to minimize blind spots in the light path through the valve.

Accordingly, said valve is configured for changing the direction of at least part of impinging light upon irradiation of the sample volume, e.g. the capillary tube or channel, which can be filled with sample, e.g. blood or urine. Impinging light may, for example, be produced by a light source as defined herein, i.e. come from an outside entity, and traverse the valve on its way to the sample volume, e.g. a capillary tube or channel. Upon impacting on the valve structure part of said light may be deviated on its way into the sample volume. For example, a percentage of said impinging light, e.g. 5%, 10%, 15% or more may change its direction and arrive at sections of the sample volume which would not have been reached without said configuration of the valve, thus allowing for an improved imaging of the sample volume and accordingly of potential filling discrepancies such as voids or air bubbles.

In a further embodiment of the invention, the light transmission through a sample may be determined in the valve or in a sub-portion of the valve of a sample container. It is particularly preferred that any imaging of the valve covers as much of the optically illuminable sections of the valve as possible. This was found to reduce the number of blind spots in the image.

In a preferred embodiment of the present invention said configuration for changing the direction of at least part of impinging light upon irradiation of the sample volume is provided by a rounded or radial edging of the valve. A sample container according to the present invention therefore preferably comprises a valve which comprises a round or radial edging. The term “round or radial edging” as used herein refers to a radius included in the bottom section of the valve. The radius may be implemented in material of the valve, preferably in a plastic material of the valve, more preferably a plastic material above a capillary tube or channel of the valve. A rounded edging or a radial edging in the valve advantageously allows the light to be bent, thus improving image quality. It is preferred that the round or radial edging is in the sector of the capillary tube or channel comprising the sample. Typically, in a valve as mentioned herein, comprising a capillary tube or capillary channel filled with sample, e.g. blood or urine, side walls of the valve tend to obscure the detector, e.g. camera, seeing or imaging the ends of the capillary tube or channel. The valve may comprise material, e.g. plastic material, above the capillary tube or channel or at its outer edges. Accordingly, a radius may be formed in said material, e.g. plastic material. Preferably, this radius may act as a prism to bend the light. This edging form or radius has been found to provide the effect that parts of a blind spot can be imaged, or that blind spots in the optical imaging of the filling state of the sample container, in particular in said capillary tube or channel, can be minimized. An example of such a radius is depicted in FIG. 5 (502).

In a further embodiment, a valve present in a sample container according to the invention comprises polished surface material. The term “polished surface material” as used herein refers to valve material whose surface texture has been smoothened by polishing it. As was found by the inventors that such polished surface material allows to avoid exacerbated light scattering by surface texture, which is considered to adversely affect the image quality. The polishing of surface material thus advantageously reduces light scattering and thus improves image quality and reduces the maximum area of blind spots due to increased optical detection. The polished surface material is preferably present in the sector of the capillary tube or channel of the valve. The smoothening process envisaged depends on the material used. Preferably, injection moulded plastic material is subjected to highly polishing of the area of the mould above the capillary tube or channel. This advantageously results in a highly polished plastic material avoiding exacerbated light scattering by surface textures.

In a further embodiment, a valve present in a sample container as defined herein above is transparent. The term “transparent” as used herein refers to the valve's property to transmit light. Typically, the valve walls are made of a transparent material, thus allowing the transmission of light from a light source. The transparence may be a transparency of at least 50%, at least 60%, at least 70%, at least 80%, at least 90%, at least 91%, at least 92%, at least 93%, at least 94%, at least 95%, at least 96%, at least 97%, at least 98%, at least 99%, or 100%, i.e. at least 50%, at least 60%, at least 70%, at least 80%, at least 90%, at least 91%, at least 92%, at least 93%, at least 94%, at least 95%, at least 96%, at least 97%, at least 98%, at least 99%, or 100% of the light entering a valve which is empty or not filled with a sample, exits said valve.

In order to achieve such a transparency, the valve is made of suitable material. Such material preferably includes transparent plastics or glass. It is particularly preferred using transparent polycarbonate plastics. Polycarbonate is typically a very durable material. It may further be combined with coatings, e.g. a hard coating increasing its scratch-resistance. Examples of employable polycarbonate plastics include polyallyldiglycolcarbonat (PADC) or CR-39 or derivatives thereof.

In specific embodiments of the present invention a scanning means provided in a device as defined herein above, for instance a CMOS sensor or CCD image sensor may be configured to be maneuverable in or moveable into a valve or into a part of a valve present in a sample container as defined herein above. It is particularly preferred that a device is provided comprising a small format CMOS sensor or camera which is maneuverable in or moveable into a part or the valve or into the valve present in a sample container as defined herein above. Such a moveable CMOS sensor or camera may advantageously extend the field of vision and accordingly exclude or reduce blind spots in an image of the sample volume in a sample container as define herein. Thereby the probability of detecting filling state problems, e.g. the presence of voids, is increased. The valve present in a sample container according to the present invention may accordingly be configured to receive a CMOS sensor or camera and/or to allow its movements.

The provision of a small format CMOS sensor or camera configured to be maneuverable in or moveable into a part or the valve or into the valve may be combined with other features of a valve or device as mentioned herein. For instance, a lens may be provided in the device or in the sample container in order to allow further focusing. Moreover, valve structures such as round edgings or the presence of a radius may be provided in order to extend the field of vision and accordingly reduce or exclude blind spots in an image. In addition, the valve may be provided in a transparent form, as mentioned herein.

In a further aspect the present invention relates to a system comprising a device wherein the sample volume is comprised in a separable sample container as defined herein above and a sample container as defined herein above. Accordingly, a device comprising a light source, detector and assessment unit may be structurally separated from a sample container as defined herein, both entities giving rise to a system for the analysis of sample material. The system may, for example, comprise structurally separated entities such as an analyzing or reaction unit; and a reader and/or control unit.

It is preferred that the sample container as defined herein is or is comprised in a cartridge or analyzing unit and that a light source, detector element and assessment unit as defined herein above or below is comprised in a reader or control unit. It is particularly preferred that said cartridge is configured to be processed by said reader or control unit.

For example, the analyzing unit or cartridge may comprise the sample to be tested, all required reagents for a testing and all physical equipment necessary for carrying out the testing of a sample. The reader or control unit may, for example, provide pneumatic and/or electrical connections to the analyzing or reaction unit allowing to induce in the analyzing or reaction unit sample movements, reactions with the sample, measurement of parameters etc. Furthermore, the reader or control unit may comprise sensors or detecting elements allowing to accumulate measured values in the analyzing or reaction unit. Furthermore, light source(s), detector elements and assessment units necessary for the detection of the filling state of the cartridge are provided by the reader and control unit. Both elements, i.e. a reader and control unit; and an analyzing or reaction unit (cartridge) may be connected in a push fit fashion, e.g. as cradle and plug-in module. The reader may accordingly be provided with opening or receptacle structures, allowing the connection or introduction of a cartridge. The physical separation of both entities or elements of the system according to the present invention provides the advantage that the same device or reader may be used for multiple analyses, while the sample container or cartridge may comprise disposable, non-reusable or non-expensive elements such as chemical reactants or assay components etc. A sample container or cartridge according to the present invention is in a preferred embodiment thus envisaged as a single use or disposable product.

It is particularly preferred that the control unit, i.e. reader, contains openings or entrance holes in order to allow insertion of an analyzing or reaction unit, i.e. sample container or cartridge, which may be provided with lids, caps or closure heads. In further embodiments, a control unit, i.e. reader, may be provided in a light tight form. The control unit, i.e. reader, may accordingly be configured to eliminate the entrance of ambient light. For example, openings or entrance holes may be shut light tight, thus allowing the performance of filling control and further analytical steps in the absence of ambient light.

In a further embodiment the sample material is a bodily fluid. The term “bodily fluid” as used herein refers to include whole blood, serum, plasma, tears, saliva, nasal fluid, sputum, ear fluid, genital fluid, breast fluid, milk, colostrum, placental fluid, amniotic fluid, perspirate, synovial fluid, ascites fluid, cerebrospinal fluid, bile, gastric fluid, aqueous humor, vitreous humor, gastrointestinal fluid, exudate, transudate, pleural fluid, pericardial fluid, semen, upper airway fluid, peritoneal fluid, liquid stool, fluid harvested from a site of an immune response, fluid harvested from a pooled collection site, bronchial lavage, and urine. In further embodiments also material such as biopsy material, e.g. from all suitable organs, e.g. the lung, the muscle, brain, liver, skin, pancreas, stomach, etc., a nucleated cell sample, a fluid associated with a mucosal surface, hair, or skin may be tested. For such a testing, the material is typically homogenized and/or resuspended in a suitable buffer solution. In further additional embodiments, samples from environmental sources, e.g. water samples, meat or poultry samples, samples from sources of potential contamination etc. may be used. Such samples may also be processed in order to liquefy them, e.g. by homogenization and/or dissolution in a buffer. Such a homogenization and resuspension in a suitable buffer may also be used in case of non-liquid stool samples, e.g. in solid feces samples.

In further embodiments bodily fluid or sample material as mentioned herein above may be processed by adding chemical or biological reactants. This may be performed in order to stabilize the sample material, to remove sample components, or to avoid interaction in samples. For example, EDTA or heparin may be used to stabilize blood samples.

It is particularly preferred using blood, i.e. whole blood, or urine samples. For other samples the light source(s), e.g. the emitted wavelengths, and detector elements etc. may have to be adjusted in order to allow the determination of absorbance of light by the sample.

In another aspect, the invention relates to a method for assessing the fill level of a sample volume, wherein said sample volume is configured for holding sample material, comprising:

irradiating the sample volume with light having a wavelength of 475-575 nm or 260-350 nm;

detecting light transmitted through the sample volume in response to said irradiation; and

assessing the fill level of the sample volume based on the detected light.

The elements mentioned in the context of the method reflect the elements of a device as described herein above. Thus, the above provided definitions apply accordingly.

The irradiation of a sample volume therefore implies irradiating a sample with a wavelength of 475-575 nm or 260-350 nm, e.g. light having a wavelength of 475 nm, 480 nm, 485 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm or any wavelength in between the indicated values, or a sub-range of the mentioned range, e.g. 500-560 nm, 510-550 nm, 520-550 nm, 530-550 nm, 500-530 nm, 500-540 nm etc., or with light having a wavelength of 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm or any wavelength in between the indicated values or a sub-range of the mentioned range, e.g. 270-330 nm, 280-340 nm, 290-350 nm, 300-350 nm, 310-350 nm, 320-350 nm, 260-300 nm, 260-310 nm etc.

Detecting light transmitted through the sample accordingly means that the transmittance and/or absorbance of light by a substance in the light path, e.g. by the sample such as blood or urine may be registered in a detector unit. The detection may, in specific embodiments, comprise the detection of specific wavelengths, or wavelength ranges. The irradiation and detection may further comprise additional steps such as diffusing the light after emittance from the light source, channeling the light through optical fibers, focusing light by using apertures and/or lens systems etc. as described herein. The detection may particularly be performed by using a small format CMOS camera with a wide angle lens which is moveable inside of a valve structure of a device as described herein.

Subsequent to the detection, one or more assessment steps may be carried out comprising the calculation of the degree of transmittance of the light through the sample and a comparison of the result with suitable control values. Subsequently, a comparison results may be further compared with threshold values indicating whether the measured value reflects a correct filling, or an incorrect filling, e.g. due to the presence of voids or air bubbles. Threshold values which may be used in this context have been defined herein above.

In further embodiments of the invention the method as outlined above may comprise one or more additional steps. In one embodiment, a detection of an incorrect filling state subsequent to the assessment may lead to an interruption, stopping or pausing of the assay testing activities of the device, e.g. the device does not continue with sample analysis procedures such as cell counting, measurement of temperature or pH etc. Additionally, or alternatively, the assessed information on the filling state of the sample volume, in particular information on an incorrect filling state of the sample volume, may be displayed to a user or patient. The user or patient may, in a specific embodiment, be requested to repeat the sample taking or to remove and/or replace a cartridge detected to be incorrectly filled.

In further embodiments, the method may comprise a transmission step to a user interface, and/or to a hospital or medical practice patient record system. Additionally or alternatively, data may be transferred from such a practice or hospital patient record system to a reader unit in order to obtain suitable control values for sample parameters. For example, in case an incorrect filling state has been detected, the device, e.g. reader, may transfer a request to a user interface and/or practice or hospital patient record system for transmission of personal medical data of the user or patient. Such personal medical data may, for example, comprise data on previously known diseases, or previously obtained information on bodily fluids acquired from the patient. Such data may subsequently be compared with the information relating to the filling state of the sample volume and may be used to decide whether the detection of an incorrect filling state of a sample volume is due to voids such as air bubbles or may be caused by a medical condition of the patient. In case such a medical condition is found to be a potential explanation for a detected incorrect filling state, the analysis of the bodily fluid may be continued. Additionally, information on the performed comparison may be transferred to a practice or hospital patient record system, and/or may be saved in a personal patient archive in the device, e.g. reader, or a user interface, or a patient record system.

In a further embodiment the invention relates to a process for performing a filling state detection in a device as mentioned herein, comprising one or more of the following steps:

filling of a sample volume, e.g. present in a sample container or cartridge, with a bodily fluid, e.g. blood or urine; this filling may be performed in a bathroom or at the bedside;

allowing the insertion of a sample container, e.g. cartridge into a reader, e.g. by opening a lid; the patient may accordingly be prompted by an indication on a display to insert a sample container or cartridge;

detecting the presence of the sample container, e.g. cartridge;

moving the sample container, e.g. cartridge, to a suitable position (position 1) for further activity, e.g. entirely entered into a reader;

closing of the reader in order to eliminate ambient light;

detecting the filling state of the sample in the sample container, e.g. cartridge; this may be carried out in the previous position (position 1);

removing of the sample container, e.g. cartridge, upon completion of the filling detection state (e.g. in case an incorrect filling is detected), or moving forward of the sample container to a further position in the reader (position 2) allowing for the performance of additional analytical steps, e.g. the performance of assays;

upon arriving at position 2 in the reader actuating a pin, e.g. a vertical steel pin, in a valve within the cartridge to yield valve movements;

after completion of valve movements, it is possible drawing the sample, e.g. blood or urine, into further sectors, chambers or zones of the cartridge, e.g. sectors comprising diluents, reactants, sensors, reaction zones etc.:

starting of performance of assays in said additional sectors of the cartridge.

These steps may be combined with further steps necessary for the operation of the device. For example, status information of the completion of a step may be transmitted to a control unit of the device. In case an interruption of the process is necessary, e.g. upon the detection of incorrect filling states, preferably a removal of the cartridge is envisaged. In specific embodiments also a transmission of a request for additional input regarding patient information with respect to the potential outcome of a filling measure is envisaged. In a further specific embodiment of the invention, the corresponding patient information may also be obtained in an earlier or first step, e.g. upon insertion of the cartridge into the reader.

The step of detecting the filling state of the sample in the sample volume, e.g. in a sample container or cartridge, or present in the device itself, may be carried out on the basis of a suitable algorithm. Such an algorithm can be any algorithm allowing the imaging of the presence of sample material and comparing it to threshold valued, which would be known to the person skilled in the art. It is preferred that the algorithm comprises at least the following steps:

acquiring an image of the sample material;

checking the focus of the image;

COM calculating a region of interest;

vertical line scanning to determine the sample material center and sample material rotation;

horizontal line scanning to determine the presence of a void;

acquiring an image of the sample material;

checking the focus of the image;

COM calculating a region of interest;

vertical line scanning to determine the sample material center and sample material rotation; and

horizontal line scanning to determine the presence of a void.

In further embodiments additional steps may be carried out:

acquiring an image of the sample material;

checking the focus of the image;

adjusting the focus of the image; this steps becomes necessary in case the image is not focused

COM calculating a region of interest;

vertical line scanning to determine the sample material center and sample material rotation;

horizontal line scanning to determine the presence of a void; the procedure end here if no void is detected.

In further embodiments additional steps may be carried out in case a void is detected:

acquiring an image of the sample material;

checking the focus of the image;

optionally adjusting the focus of the image; this steps becomes necessary in case the image is not focused

COM (centre of mass) calculating a region of interest;

vertical line scanning to determine the sample material center and sample material rotation;

horizontal line scanning to determine the presence of a void;

calculating the size and/or volume of the void.

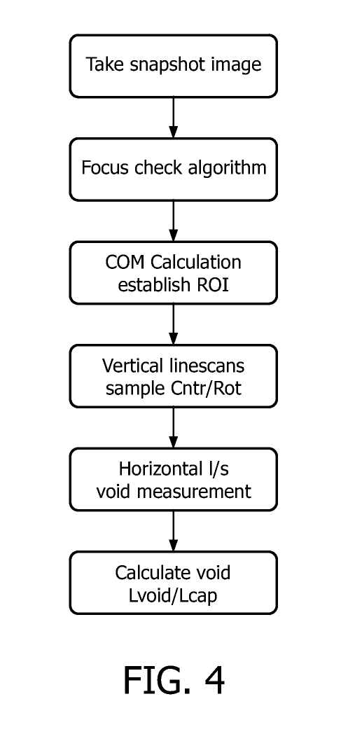

Thus, in a particular embodiment of the invention the image processing algorithm follows the flow chart depicted in FIG. 4. First a snapshot image is taken of the sample. An algorithm to check if the image is focused is then run and the focus is adjusted if necessary. A centre of mass (COM) approach is used to define the region of interest (ROI), which allows for compensation of a mechanical offset with respect to the sensor centre point. The COM determines the initial location for the first linescans that will determine the sample centre and the sample rotation. Horizontal linescans will then be performed, allowing the determination of the void, if a void is present.

Algorithms as defined herein may be provided in the form of computer programs. These computer programs may be stored or distributed on a suitable medium such as an optical storage medium or a solid-state medium supplied together with or as part of other hardware. It may also be distributed in other forms, e.g. via the internet or via wired or wireless telecommunication systems.

Upon the calculation of the size or volume of the void, e.g. air bubble, a comparison with threshold values may be performed. In particular, it may be calculated whether the detected void, e.g. air bubble, is above or below a predetermined tolerance limit. In specific embodiments, the tolerance limit for voids may be set to about 20%, 15%, 14%, 13%, 12%, 11%, 10%, 9%, 8%, 7%, 6%, 5%, or 4% of the possible filling volume of the sample volume, e.g. present in a sample container. It is preferred that the tolerance limit or threshold value be set to 10% of the possible filling volume of the sample volume. Thus, if the void or air bubble calculated exceeds the indicated tolerance limit, in case of a tolerance limit of 10% is larger than 10% of the possible filling volume of sample volume, the filing state is labeled incorrect and an interruption and/or termination of the analysis of the bodily fluid follows.

In further specific embodiments, an alternative recalculation of the fill level of the sample volume may be carried out. Such a recalculation may lead to a determination of the actual filling of the sample volume and may provide correction parameters for subsequent analytical steps. Accordingly, subsequent analytic processes in the device may be started despite an incorrect filling state of the sample volume. Obtained results may be corrected, if necessary, by the specific parameters of the recalculated overall volume of sample in the sample volume. A corresponding correction may, for instance, be based on previously obtained parameters with reduced filling states.

In a further aspect the present invention relates to a use of a device as defined herein above, or of a sample container as defined herein above, or of a system of device and sample container as defined herein above, for home-monitoring parameters of a bodily fluid of a subject. It is preferred that in particular parameters of blood or urine are home-monitored. The term “parameter of a bodily fluid” as used herein refers to any physical, physico-chemical, chemical or biological parameter which can be measured in assay approaches or by sensoring fluid samples. Examples of such parameters are pH of a sample, temperature of a sample, concentration or presence of ions, charged entities or small molecules in a sample, concentration or presence of proteins, peptides, nucleic acids, lipids, sugars or other biochemical entities in a sample, size of particles in a sample, number of particles in a sample, identity of particles in a sample, form of particles in a sample or the distribution of two or more different particles in a sample. Preferred is the detection of blood cells, e.g. white blood cells, or subgroups of leukocytes, and the determination of the amount of red blood cells or hemoglobin by using a device, sample container or system as defined herein above. The term “home-monitoring” as used herein means that by using a device, sample container or system as described herein the determination of the above mentioned parameters may be carried out at home by the user or patient and does not require the intervention or assistance of a medical professional. It is, however, not excluded by the presently claimed use that medical professionals use a device, sample container or system according to the invention, or that they assistant in its usage. The device, sample container or system may accordingly also be employed in hospital environments, medical practices, or in laboratories; it may further also be used by medical professionals, such as nurses, lab technicians or medical doctors.

In specific embodiments of the invention a device, sample container or system as described herein may be used during a chemotherapy treatment of a patient. During chemotherapy treatments patients with low blood cells counts are typically in danger of complications in form of infections, or may have difficulties in receiving a further treatment due to low cells counts. By using a device, sample container or system as mentioned herein, such patients can detect their situation earlier, and may more rapidly request suitable intervention by medical professionals. In further embodiments of the invention a device, sample container or system as described herein may be used during a Warfarin treatment of a patient for anticoagulation therapy. The home-monitoring possibilities of the device, sample container or system allow a rapid detection of coagulation problems in blood and may give rise to suitable rapid interventions. Similarly, a device, sample container or system as described herein may be used during the treatment of autoimmune diseases to home-monitor parameters of bodily fluids such as blood. An example is the treatment of rheumathoid arthritis. The home-monitoring possibilities of the device, sample container or system allow a rapid detection of coagulation problems in blood and may give rise to suitable rapid interventions. Further employments in different diseases stages or during the treatment of certain diseases, e.g. cancer treatment, treatment of cardiac disorders etc. is also envisaged.

Further embodiments of the present invention are reflected by the figures.

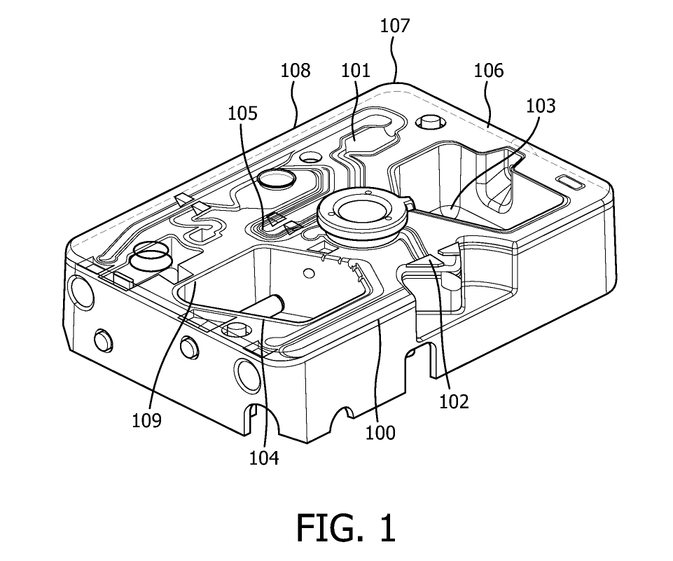

FIG. 1 schematically shows a cartridge 100 for a device comprising an automatic fill detection feature according to the present invention. The cartridge of FIG. 1 comprises a valve 101. The valve is configured to control sample movements in the cartridge. A sample is introduced into the cartridge at an application port for blood sample 103. A connection to a reader is established by interface portion 102. The cartridge of FIG. 1 further comprises pneumatic connections to the reader 104, a mixing chamber 105, a diluents reservoir 106, an overflow chamber 107, an optical measurement chamber 108, and coulter electrode connections for the reader 109.

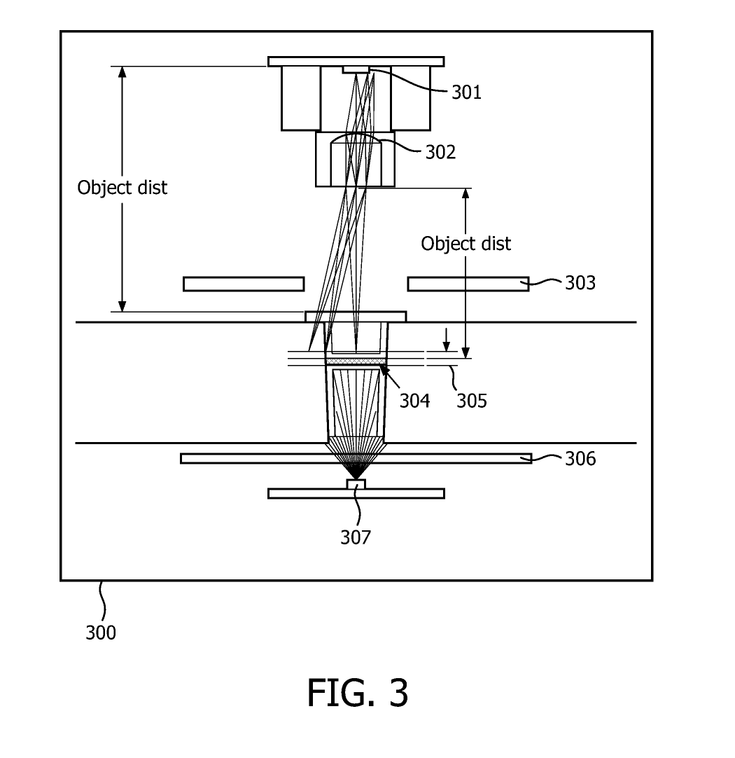

FIG. 3 schematically shows a system for automatic detection implemented in a stationary reader 300. The system comprises a light source 307, which may be comprised of green LEDs. The light source illuminates through a diffuser 306 into the valve area of an introduced cartridge comprising a sample 304 under examination. The sample area has a depth of field (DOF) 305. Transmitted light passes an aperture 303 and a lens or lens system 302, which focus the image. It is subsequently captured by a camera such as a CMOS image sensor 301.

FIG. 5 schematically shows a cross-section through a valve 500 according to an embodiment of the invention. The valve comprises a capillary tube or channel 501 which can be filled with sample, e.g. blood. The detection of air bubbles is carried out in this portion of the valve. The presence of a round edging or radius 502 allows the imaging of the capillary tube or channel without or with a reduced number of blind spots.

FIG. 6 schematically depicts an embodiment of the invention in which light 601 is being bent by a valve 500 comprising a lens 602. The light is subsequently detected by a camera 603. The filling state in a capillary tube or channel 501 can accordingly be detected.

The following example and figures are provided for illustrative purposes. It is thus understood that the example and figures are not to be construed as limiting. The skilled person in the art will clearly be able to envisage further modifications of the principles laid out herein.

### EXAMPLES

### Example 1

### Detection of Blind Spots by Different Imaging Techniques

The device described in the invention advantageously allows automatic fill detection to take place. This solution works in particular well if the air bubble or defect is in the middle of a valve. There may be, however, a part of a valve that cannot be imaged using a standard light source and detector. In order to improve the system setup, different image and lens options were tested. The tested options are indicated in Table 1, below.

Option A is standard imaging. The blind distance from the wall was found to be 12.6 to 12% on each side, i.e. greater than a 10% deviation.

Option B involves using a lens in the cartridge. The blind distance from the wall was found to be 10.5-9.8% on each side.

Option C requires the camera to be mechanically maneuvered into the valve to extend the field of vision. The blind distance from the wall was found to be 9.8 to 9.1% on each side.

TABLE 1

| Option | A - Normal Imaging | B - Field Lens | C - Wide Angle |
|---|---|---|---|
| Description | Camera set on central axis | Field lens used to locally increase | Small camera with wide angle lens |
| | with limited [symbol] iekl angle | field angle | inserted into barrel |
| Bind Distance from | −0.0-0.85 mm | −0.75-0.7 mm | −0.7-0.65 mm |
| wa [symbol] (mm & %) | (12.6%-12% each side) | (10.5%-9.8% each side) | (9.8%-9.1% each side) |
| Moving Parts | None | Field Lens | Camera |
| Camera | Standard CMOS | Standard CMOS | Small format CMOS |
| | Wide Range Available | Wide Range Available | More Limited Availability |
| Lens | Off the Shelf | Off the Shelf | May require custom lens |
| | | Custom Field Lens | |

[symbol]

indicates data missing or illegible when filed

1. A device for the analysis of sample material comprised in a sample volume, comprising:
a light source, wherein said light source is configured for irradiating the sample volume;
a detector, wherein said detector is configured to detect light from said sample volume in response to an irradiation of said sample volume by the light source; and
an assessment unit for assessing the fill level of said sample volume based on the detected light.

2. The device of claim 1, wherein said sample volume is comprised in said device.

3. The device of claim 1, wherein said sample volume is comprised in a separable sample container.

4. The device of claim 1, wherein said detector is configured for detecting light transmitted through the sample volume.

5. The device of claim 1, wherein said light source is configured to emit light having a wavelength of 475-575 nm or 260-350 nm.

6. The device of claim 1, wherein said device additionally comprises an optical element configured to image at least a part of the sample volume onto the detector, preferably a lens, a mirror, or an optical fiber.

7. The device of claim 1, wherein said device comprises scanning means configured to detect light from the entire sample volume, preferably comprising a camera.

8. The device of claim 1, wherein said assessment of the fill level of the sample volume comprises the assessment of the presence of a void such as an air bubble.

9. A sample container configured for holding sample material in a sample volume comprising a valve configured for moving said sample material, wherein said valve is configured for changing the direction of at least part of impinging light upon irradiation of the sample volume, preferably via a rounded or radial edging of the valve.

10. The sample container of claim 9, wherein said valve comprises polished surface material.

11. The sample container of claim 9, wherein said valve is transparent, preferably comprising polycarbonate.

12. A system comprising a device as defined in claim 3, preferably such that the sample container is or is comprised in a cartridge and that the light source, detector and assessment unit are comprised in a reader, wherein said cartridge is configured to be processed by said reader.

13. The device of claim 1, wherein said sample material is a bodily fluid, preferably blood or urine.

14. Method for assessing the fill level of a sample volume, wherein said sample volume is configured for holding sample material, comprising:
irradiating the sample volume with light having a wavelength of 475-575 nm or 260-350 nm;
detecting light transmitted through the sample volume in response to said irradiation; and
assessing the fill level of the sample volume based on the detected light.

15. The method of claim 14, wherein said assessment of the fill level is carried out with a image processing algorithm comprising the steps:
acquiring an image of the sample material;
checking the focus of the image;
optionally adjusting the focus of the image;
COM calculating a region of interest;
vertical line scanning to determine the sample material centre and sample material rotation;
horizontal line scanning to determine the presence of a void;
optionally calculating the size and/or volume of the void;
optionally recalculating the fill level of the sample volume.
